(19) United States
(12) Patent Application Publication
(10) Pub. No.: US 2014/0074170 A1
(43) Pub. Date: Mar. 13, 2014

(54) Delivery Device With Interior Dilation Element Channel

(71) Applicants: Herbert H. Mertens, San Diego, CA (US); Bassem A. Georgy, San Diego, CA (US); Lex Jansen, Menlo Park, CA (US); Beto Cantu, San Francisco, CA (US)

(72) Inventors: Herbert H. Mertens, San Diego, CA (US); Bassem A. Georgy, San Diego, CA (US); Lex Jansen, Menlo Park, CA (US); Beto Cantu, San Francisco, CA (US)

(21) Appl. No.: 13/371,141

(22) Filed: Feb. 10, 2012

(51) Int. Cl. A61B 17/88; A61F 2/44; A61B 17/60

(52) U.S. Cl. 606/279; 606/90; 623/17.16

(57) ABSTRACT

Described herein are devices and systems that can be used to perform minimally-invasive procedures including dilation, distraction and delivery of an implant. In one aspect, disclosed herein is a surgical access and orthopedic distraction device having a first cannula element including a first elongate portion and a first head portion; and a second cannula element including a second elongate portion and a second head portion. The first and second head portions reversibly mate to define a lip surrounding at least a portion of a proximal access portal of an elongate, tubular working channel formed by the first and second elongate portions.

### BACKGROUND

The spinal column includes, among other structures, the bony vertebrae which surround the spinal cord, and the intervertebral discs. In a healthy spine, the discs maintain separation between the vertebrae, promote fluid circulation throughout the spine, and provide a cushioning effect between the bony vertebral structures. A variety of conditions can affect both the vertebrae and intervertebral discs.

Due to the elastic nature of an intervertebral disc, the disc is subject to injury if the disc becomes overstressed, for example, by trauma to the spine, excess body weight, improper mechanical movements and the like. Intervertebral disc injuries and other abnormalities result in serious back pain and physical disability and are often chronic and difficult to treat. Such abnormalities include, but are not limited to, localized tears or fissures in the disc annulus, localized disc herniations with contained or escaped nuclear extrusions, and circumferential bulging discs. Discs also experience degeneration over time which can accelerate these problems.

One of the common disc problems that occur when the entire disc bulges circumferentially about the annulus rather than in specific, isolated locations. This may occur for example, when over time, the disc weakens, bulges, and takes on a “roll” shape. The joint may become unstable and one vertebrae may eventually settle on top of another. This problem typically continues to escalate as the body ages, and accounts for shortened stature in old age. Osteophytes may form on the outer surface of the disc and further encroach upon the spinal canal and nerve foramina. This condition is called spondylosis.

Traditional non-surgical treatments of disc degeneration and abnormalities include bed rest, pain and muscle relaxant medication, physical therapy or steroid injection. Such therapies are directed primarily at pain relief and potentially delaying further disc degeneration. Eventually, most non surgical treatments fail In many cases surgical alternatives, often spinal fusion, may be the only option. Spinal fusion methods are aimed at causing the vertebrae above and below the injured disc to grow solidly together forming a single piece of bone. This procedure is carried out with or without discectomy (surgical removal of the disc). Another procedure, endoscopic discectomy, involves removing tissue from the disc percutaneously in order to reduce the volume of the disc, thereby reducing impingement of the surface of the disc on nearby nerves.

To deliver implants and perform procedures within and around the disc space, it can be necessary to separate and create an access channel to the disc space. One procedure uses a series of successively larger dilators to separate muscle fibers of the spine to create a pathway to the disc space. The dilator kits are generally inserted one over the other which can pose a risk to adjacent spinal nerves and other sensitive tissues in the location of dilator placement.

### SUMMARY

The subject matter described herein provides many advantages. For example, the current subject matter is a device that can perform three key functions for minimally-invasive procedures including dilation, distraction and delivery of an implant.

In one aspect, disclosed herein is a surgical access and orthopedic distraction device having a first cannula element including a first elongate portion and a first head portion; and a second cannula element including a second elongate portion and a second head portion. The first and second head portions reversibly mate to define a lip surrounding at least a portion of a proximal access portal of an elongate, tubular working channel formed by the first and second elongate portions. The device also includes a first, rigid dilator element having an outer diameter larger than the inner diameter of the working channel. The first, rigid dilator element is configured to be inserted through the working channel and separate the first cannula element a distance from the second cannula element and increase an inner diameter of the working channel to distract adjacent bones away from one another in at least a first direction.

The device can further include a third elongate portion and a fourth elongate portion. Separation of the third elongate portion a distance from the fourth elongate portion can enlarge the inner diameter of the working channel and distract the adjacent bones away from one another in at least a second direction. The adjacent bones can be a superior and an inferior vertebral body. The device can further include a second, rigid dilator element having an outer diameter larger than the inner diameter of the working channel and an inner diameter larger than the outer diameter of the first, rigid dilator element. The device can further include an expansion element mechanically coupled to one or both of the first head portion and the second head portion. The expansion element can be configured to provide auxiliary separation of the first cannula element from the second cannula element. The device can further include a handle coupled to at least one of the first head portion or the second head portion and configured to manipulate the device. The first and second elongate portions can have a U-shaped cross section and the working channel can have a rectangular cross section. The lip surrounding at least a portion of the proximal access portal can include a beveled edge. The first and second elongate portions can be radiopaque.

In another aspect, disclosed is a method of treating a spinal disorder in a patient including accessing a disc space between an inferior vertebra and a superior vertebra with a trocar inserted through Kambin's triangle; feeding an access device over the trocar to the disc space; sequentially advancing dilator elements through the working channel of the access device; separating the first elongate portion away from the second elongate portion; pressing against the endplates of the inferior and superior vertebrae with a distal end region of the first and second elongate portions; distracting the superior and inferior vertebrae away from one another with the distal end region of the first and second elongate portions; and dilating tissues surrounding the first and second elongate portions of the access device. The access device can include a first cannula element having a first elongate portion and a first head portion; and a second cannula element having a second elongate portion and a second head portion. The first and second head portions reversibly mate to define a lip surrounding at least a portion of a proximal access portal of an elongate, tubular working channel formed by the first and second elongate portions.

Sequentially advancing dilator elements can include inserting a first, rigid dilator element through the working channel of the access device and inserting a second, rigid dilator element through the working channel over the first, rigid dilator element. The first, rigid dilator element can have an outer diameter that is larger than an inner diameter of the working channel. The second, rigid dilator element can have an outer diameter larger than the inner diameter of the working channel and an inner diameter larger than the outer diameter of the first, rigid dilator element. Inserting the first, rigid dilator element through the working channel can separate the first and second elongate portions a distance away from one another. Inserting the second, rigid dilator element through the working channel over the first, rigid dilator element can enlarge the distance away the first and second elongate portions are separated from one another. Enlarging the distance away the first and second elongate portions are separated from one another can enlarge the inner diameter of the working channel. The method can further include using the distal end region of the first and second elongate portions to prepare the endplates for fusion. The method can further include delivering an intervertebral fixation device through the working channel of the access device to the disc space. The method can further include injecting bone growth stimulating material into an internal volume of the intervertebral fixation device.

In another aspect, disclosed is a kit for use in accessing and distracting tissue including an access and distraction device and a first, rigid dilator element having an outer diameter larger than an inner diameter of the working channel. The access and distraction device includes a first cannula element having a first elongate portion and a first head portion; and a second cannula element having a second elongate portion and a second head portion. The first and second head portions reversibly mate to define a lip surrounding at least a portion of a proximal access portal of an elongate, tubular working channel formed by the first and second elongate portions. The first, rigid dilator element is configured to be inserted through the working channel and separate the first cannula element a distance from the second cannula element and increase an inner diameter of the working channel to distract adjacent bones away from one another in at least a first direction.

The kit can further include a second, rigid dilator element having an outer diameter larger than the inner diameter of the working channel and an inner diameter larger than the outer diameter of the first, rigid dilator element. The kit can further include an intervertebral fixation device deliverable through the working channel of the access device to a disc space. The kit can further include a delivery device coupled to the intervertebral fixation device. The kit can further include bone growth stimulating material.

More details of the devices, systems and methods for dilation, distraction and delivery of implants are set forth in the accompanying drawings and the description below. Other features and advantages will be apparent from the description and drawings, and from the claims. It should be appreciated that although the devices, systems and methods described herein are described in reference to spinal procedures that they need not be limited to the spine. The devices, systems and method described herein can be used in non-spine procedures such as other orthopedic procedures, vascular, urological and gastroenterological procedures, or other non-spine procedures.

### BRIEF DESCRIPTION OF THE DRAWINGS

These and other aspects will now be described in detail with reference to the following drawings. Generally speaking the figures are not to scale in absolute terms or comparatively but are intended to be illustrative of claimed features. Also, relative placement of features and elements may be modified for the purpose of illustrative clarity.

FIG. 1A is simplified, sagittal view of a vertebrae pair;

FIG. 1B is a simplified, sectional axial view of a vertebra;

FIG. 2 is a simplified, sagittal view of a vertebrae pair illustrating Kambin's Triangle;

FIG. 3 is a perspective view of an access device positioned between a vertebrae pair;

FIGS. 4A-4D are perspective, schematic views of the access device being expanded inside out by two dilators;

FIG. 5 is a posterior-lateral view of the access device of FIG. 3;

FIG. 6 is a lateral view of the access device of FIG. 3 delivering an implant between the vertebrae pair;

FIG. 7 is a posterior-lateral view of a pair of implants positioned within the disc space of the vertebrae pair.

### DETAILED DESCRIPTION

Described herein are devices, systems and methods for minimally-invasive access to the treatment regions, such as the disc space. In particular, described are devices for the posterior lateral approach using a set of dilators delivered through an expandable access device into the disc space and expanded through inside-out dilation to move and protect adjacent spinal nerves. It should be appreciated that the term “access device” is not intended to be limited insofar as the functions the device can perform.

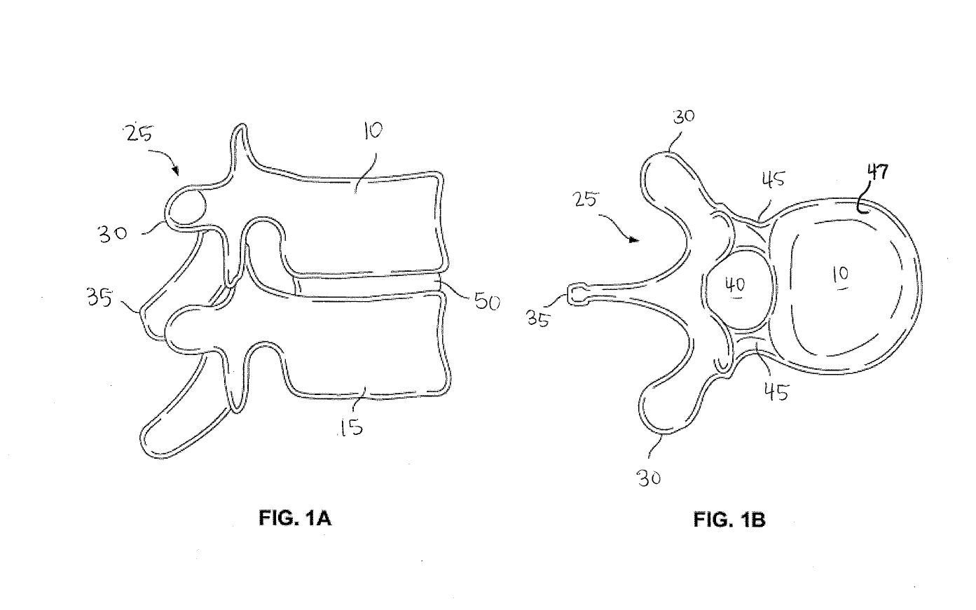

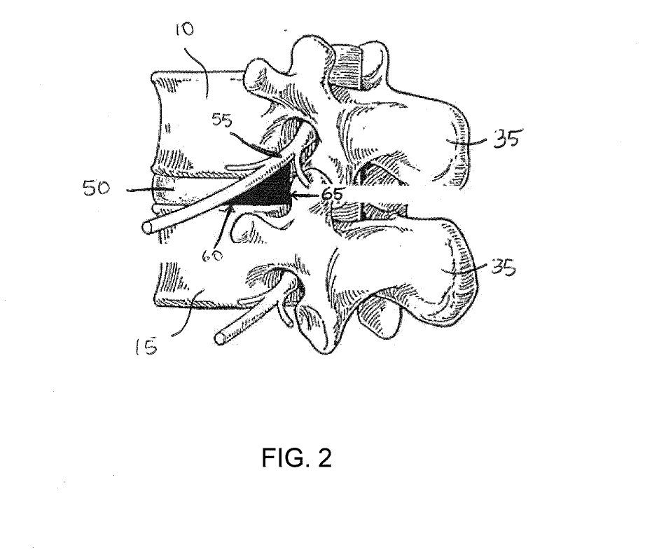

FIG. 1A is a simplified sagittal view of a vertebrae pair 10, 15. FIG. 1B is a simplified, sectional axial view of the vertebrae 10 of the vertebrae pair shown in FIG. 1A. Each vertebra 10, 15 includes lamina 25, transverse processes 30, a spinous process 35, central canal 40, and pedicles 45. A disc 50 comprised of an annulus and disc nucleus (not shown) is located between the vertebrae pair 10, 15 where the vertebrae pair 10, 15 and disc 50 form a coupled articulated jointed bony interface at the location of a hardened outside endplate 47 of the vertebrae.

The devices, systems and methods described herein are generally designed for minimally-invasive procedures including “mini-open” procedures characterized by small incisions, percutaneous procedures, or a combination thereof. The devices, systems and methods described herein can be deployed through a variety of access channels. It should be appreciated that the devices described herein can be inserted laterally, anteriorly, posteriorly and/or posterior-laterally. The devices, systems and methods described herein are generally used under fluoroscopic guidance. It should be appreciated that although the devices, systems and methods described herein are described in reference to spinal procedures that they need not be limited to use in the spine. The devices, systems and method described herein can be used in non-spine procedures such as other orthopedic procedures, urological and gastroenterological procedures, or other non-spine procedures.

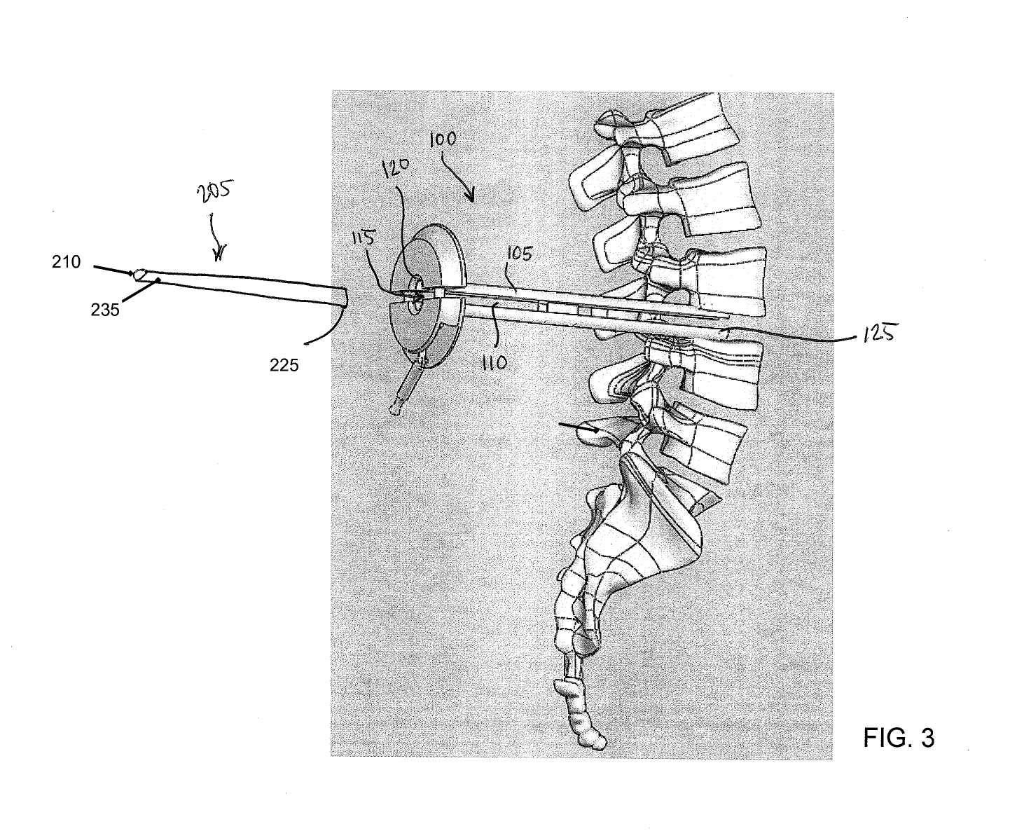

As will be described in more detail below, instrumentation described herein can be introduced between the traversing and exiting nerves in an area known as Kambin's Triangle (see FIG. 2). Kambin's Triangle is a site of surgical access for posterolateral discectomy or intervertebral fusion as described herein. Kambin's Triangle is formed over the dorsolateral disc with the hypotenuse being the exiting root 55 (the root that leaves the spinal canal just cephalad (above) the disc, the base being the superior border 60 of the caudal vertebra 15, and the height being the traversing nerve root 65 (the root that leaves the spinal canal just caudad (below)). The vital structures involved with the posterior or posterior/lateral approaches are the nerve roots. The triangle can be enlarged by retracting the traversing nerve root medially. If retraction is done too vigorously, however, retraction injuries may occur and serious complications such as nerve root sleeve tear may result, causing spinal fluid leakage, nerve root injury, avulsion and even spinal cord injury. The devices described herein can be used to expand the space with the Triangle and/or other approaches where nerve and nerve roots are present and it is desirable to avoid or push to the side to obtain access to the spine or deliver an implant or bone graft material without posing a risk of injury to these delicate structures.

The devices described herein can be used independently or in conjunction with other systems or devices to provide stability to the spine. For example, the access devices described herein can be used to deliver an implant as described in co-pending application serial no. [ATTORNEY DOCKET NO. 42144-502F01US], entitled “Implant With Outwardly Extending Fixation Elements,” filed on the same day herewith, and which is incorporated by reference in its entirety.

The figures illustrate the anatomic landmarks in the spine and access through the vertebrae to the intervertebral disc space in schematic. Those skilled in the art will appreciate that actual anatomy include anatomical details not shown in the figures. It should also be appreciated that although the drawings illustrate the devices implanted in the thoracic vertebrae, that the devices and methods can be used along entire spine including cervical, thoracic and lumbar levels.

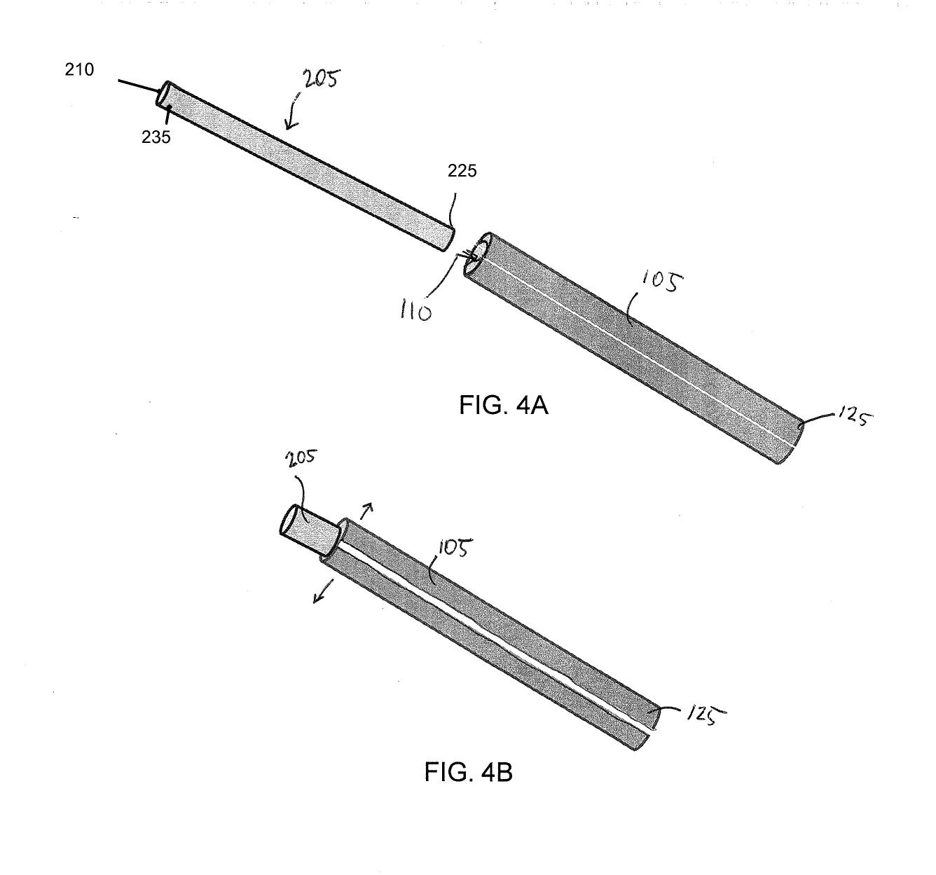

FIG. 3 illustrates an example of an access device 100 that can be interiorly expanded or dilated by one or more rigid dilator elements 205. The access device 100 generally includes opposing, separable elongate jaws 105. The use of the term jaw is not meant to be limiting, but rather to provide a description of opposing and separable elongate elements. It should also be appreciated that although the figure illustrates a pair of jaws that more than two opposing elements can be incorporated. For example, three, four, or more jaws are to be considered herein. Each jaw 105 can have a proximal head portion 135 and a distal elongate tubular portion that can reversibly mate or couple together to form at a distal region an elongate tubular body surrounding an interior lumen 110 and at a proximal region an enlarged proximal head portion or lip that surrounds at least a portion of a proximal access portal into a working channel of the elongate tubular body. It should be appreciated that the cross-sectional shape of the access device 100 can vary. In an embodiment, the elongate portions of the jaws 105 are U-shaped such that the cross-sectional shape of the elongate tubular body is rectangular. It should be appreciated that the cross-sectional shape need not be rectangular and other shapes are considered herein including circular, oval, square, polygonal and other appropriate cross-sectional shape. In one example, there are four elongate portions of the jaws 105 that each form an angle or curve to create the working channel of the elongate tubular body. It should also be appreciated that the proximal head portion 135 of each jaw 105 can couple together by a variety of mechanisms. In an example, the lip or proximal head portion 135 of one jaw 105 can have a male mating element that reversibly inserts into a corresponding female mating element in the proximal head portion 135 of the opposing jaw 105. In the example where there are four jaws, each of the jaws can include a proximal head portion 135 that can couple together.

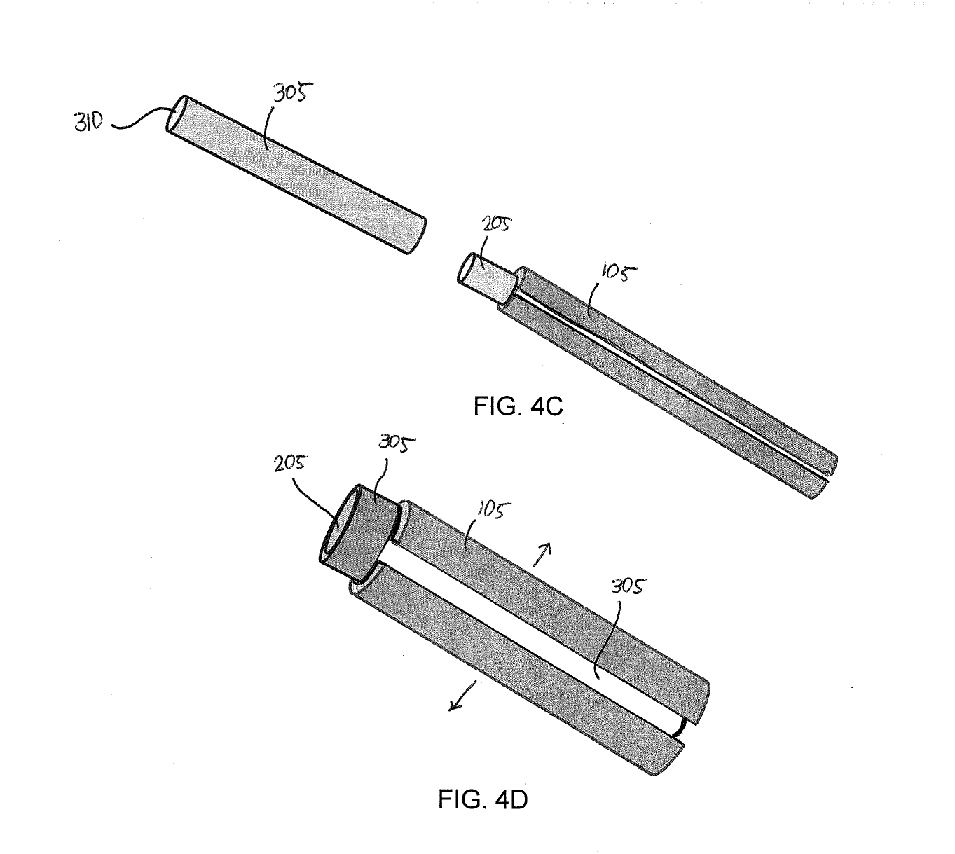

As best shown in FIGS. 4A-4D, the dilator element 205 can be a generally elongate, relatively rigid structure that can have an outer diameter that is larger than an inner diameter of the interior lumen 110 of the access device 100. Upon insertion of the larger diameter dilator element 205, the opposing jaws105 of the access device 100 can separate a distance from one another forming a larger inner diameter of the interior lumen 110 (see arrows in FIG. 4B). In an example where there are four jaws, the separation can be tailored. For example, the separation of two jaws can be performed without a separation of the other two jaws. As such, a more custom separation in more than a single direction can be achieved.

The dilator element 205 can have a cross-sectional shape that corresponds to the shape of the access device 100 and can thus, vary in shape accordingly. Generally, the dilator element 205 can have an internal lumen 210 extending from a proximal end 235 to a distal end 225 of the dilator element 205. As shown in FIG. 4C, a second dilator element 305 having an inner diameter larger than the outer diameter of the previously inserted first dilator element 205 such that the second dilator element 305 can be inserted over the first dilator element 205. Further, the second dilator element 305 can have an outer diameter larger than the inner diameter of the interior lumen 110 of the access device 100. As such, the second dilator element 305 can be wedged between the outer diameter of first dilator element 205 and the inner diameter of the interior lumen 110 of the access device 100 to further separate the opposing jaws 105 of the access device 100. The first dilator element 205 can then be withdrawn and removed through the internal lumen 310 of the second dilator element 305. It should be appreciated that disc resection and/or delivery of an implant to the disc space can be performed through the internal lumen of the last and largest dilator element inserted through the access device 100. Alternatively, the last and largest dilator element can be removed such that the access device 100 alone remains in place. In one example, the access device 100 can have more than two jaws and the dilator element 205 can have an outer diameter that is non-uniform. In this example, the dilator element 205 can be inserted with a particular orientation so as to separate one jaw or a pair of jaws away from the other.

Any number of increasingly larger dilator elements can be inserted between the previously inserted dilator element and the interior lumen 110 of the access device 100 to effect the gradual separation of the opposing jaws 105 and in turn, dilate and retract the surrounding tissues. This forms a tissue channel leading to the target implantation site in a safer gradual inside-out sort of expansion of the access device 100. Neighboring nerves and nerve roots can be protected from damage related to the advancement of the dilator elements, implants or any other materials such as allograft materials or tools inserted into the access device 100. The set of dilator elements can open the access device 100 between about 4 mm to about 14 mm for disc procedures or up to about 20 mm for vertebral applications.

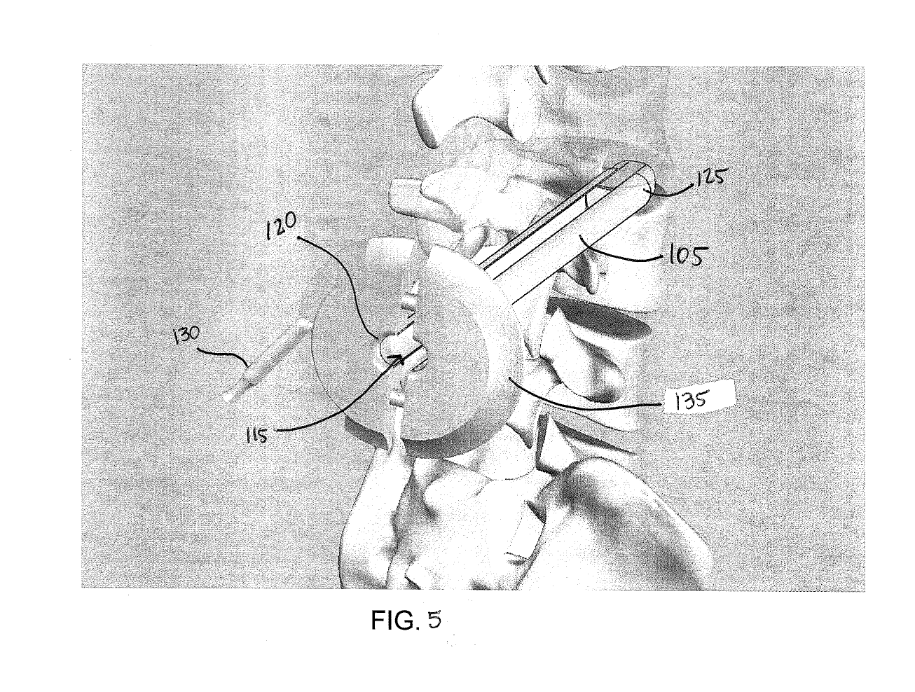

FIG. 5 illustrates the proximal end 135 of the access device 100. Proximally, the access device 100 can have an access portal 115 to the interior lumen 110 through which a distal end 225 of a dilator element 205 can be inserted. The lip surrounding at least a portion of the access portal 115 can have a beveled or rounded edge 120 to provide for a smoother insertion of the dilator element 205 through the portal 115 into the interior lumen 110. Further, the proximal end 135 of the access device 100 can have a handle 130 or other user manipulation device to assist the user in manipulating and positioning the access device 100, such as for example inserting the access device 100 over a trocar or other element introduced into the disc space. The proximal end of the access device 100 can also include an expansion element or mechanism that provides additional or auxiliary distraction/dilator force to the opposing jaws 105 of the access device 100 such as a lead screw, ratchet, crank mechanism or other feature mechanically coupled to one or both proximal head portions that will provide auxiliary separation of the opposing jaws 105 of the access device 100.

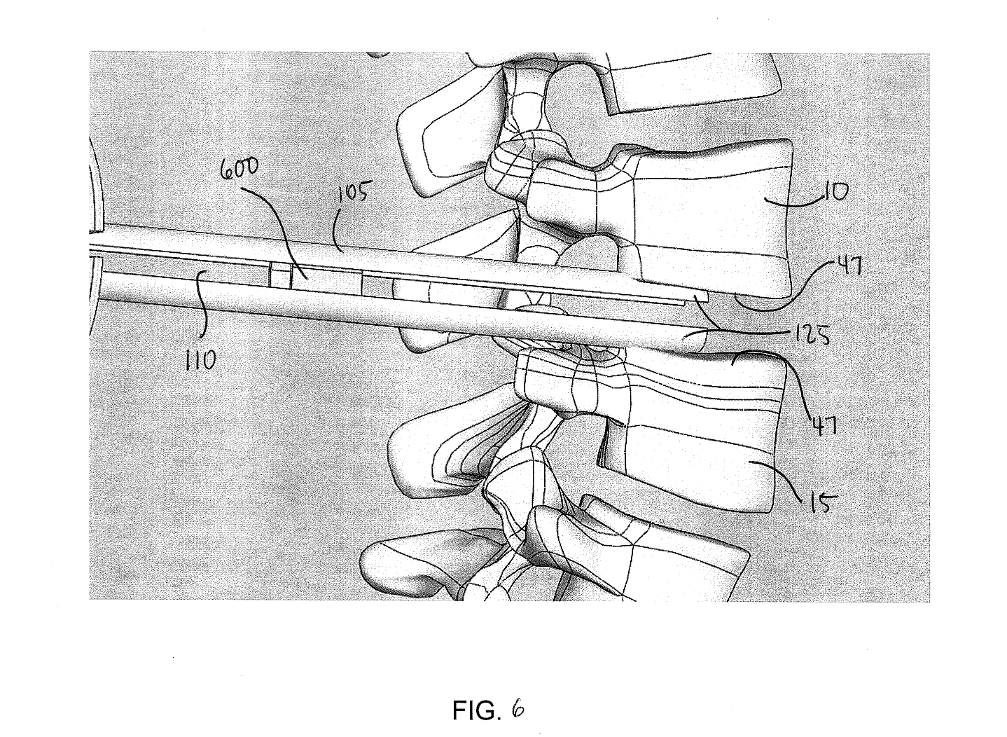

FIG. 6 illustrates the distal end 125 of the access device 100 where the opposing jaws 105 are separated and the interior lumen 110 is visible. The dilator elements and trocar are not shown. An implant device 600 is shown being delivered through the interior lumen 110 of the access device 100. The distal end 125 of each of the opposing jaws 105 can contact the vertebral endplates 47 of either the superior 10 or inferior 15 vertebrae, which in addition to applying distraction forces (which can itself also induce fusion), can prepare the vertebral pair for further fusion with the implant device 600. It should be appreciated that a separate curette or other instrument could, but need not, be used with the disclosed system to prepare the endplates 47 for fusion.

The components of the devices described herein can be composed of various materials, including stainless steel, radiolucent plastics or polymers such as PEEK, and/or relatively inert implantable materials such as titanium and titanium alloys. The material is generally radiopaque such that it can be visualized under fluoroscopy.

The devices described herein can be provided in a kit. For example, the access device 100 can be provided with one or more dilators, trocars, or other device used in the method. The kit can also include an implant or other fusion device, for example a fusion device in a pre-deployed state with a delivery device pre-loaded therein. The kit can also be packaged with instructions for use and in a format convenient for surgical operating rooms, for example, in a box or in a sterile plastic wrapping or pouch, which can be sealed and sterilized. Different types of kits are contemplated herein and can be tailored to meet the needs of a particular surgical method.

Methods of Use

It should be appreciated that each component of the systems described herein can be inserted in a variety of approaches including posterior, lateral, anterior and posterior lateral approaches. In an embodiment, the approach is posterior lateral and the devices are advanced through Kambin's Triangle. The systems and devices described herein can be used create and maintain a space for performing full or partial removal and replacement of a disc, stabilizing and fusing bony structures, or annular repair.

Generally, the patient can be in a prone position and under anesthesia that can be intravenous sedation, local, or general anesthesia. Wilson frames or pillows can be used under the patient's stomach to “open up” the spine/disc space. The frames/pillows can be repositioned or removed throughout various steps of the procedure in order to “open up” other portions of the patient's spine. For example, if the anterior part of the spine needs to be “opened up” the pillows/frames may be removed or repositioned.

A surgeon can create an incision in the back of a patient through which a trocar can be advanced through Kambin's triangle. The trocar can be advanced up to and/or into the disc space. The access device 100 can be advanced over or through the trocar and positioned through Kambin's triangle and into the disc space. At this stage, a full or partial discectomy can be performed according to methods known in the art. The access device 100 can be expanded in one or more orientations by sequentially advancing dilator elements through the interior lumen 110 of the access device 100 as described above. Each of the dilator elements has an outer diameter that is larger than the previously inserted dilator element such that they gradually separate the opposing jaws 105 of the access device 100. As the access device 100 expands, the opposing jaws 105 press against adjacent tissues creating an access channel to the disc space. Further, the expansion of the access device 100 near the distal end 125 presses against the superior and inferior vertebrae 10, 15 causing distraction of the vertebrae away from one another to achieve an optimal disc height. The access device 100 can have two, three, four or more mating components such that the inner diameter of the working channel can be expanded in a tailored fashion (i.e. upwards, downwards, sideways, etc.) As such the access device 100 described herein can both dilate and distract tissues. It should be appreciated that the term “access device” is not meant to be limiting insofar as the functions the device performs.

Once the optimal position and disc height is achieved, one or more implants 600 can be delivered through the access device 100 and deployed within the disc space. The endplate 47 of each vertebrae 10, 15 can be prepared prior to implant delivery to improve fusion of the bones with the implant, such as by contacting the distal end 125 of the device 100 against the endplates. The vertebrae 10, 15 can be prepared for fusion by scraping, breaking through, or cutting into, the endplates to allow the interposed bone graft to come into direct contact with the more vascular cancellous (spongy) bone, and to thereby urge the body to heal this induced and controlled injury to the bone such that the superior and inferior vertebrae 10, 15 become one continuous segment of bone.

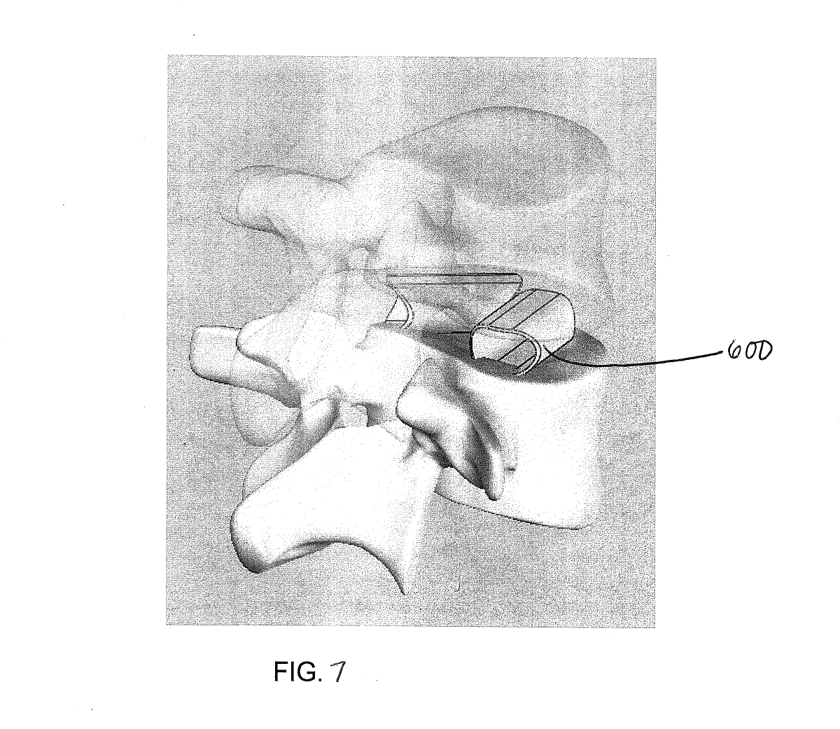

Once the implant(s) 600 is appropriately positioned and deployed through the access device 100, the access device 100 can be removed as shown in FIG. 7. The success of the steps can be confirmed by fluoroscopy prior to removal of tools and closure of the incision. The implant 600 and its deployment are described in more detail in co-pending application ser. no. [ATTORNEY DOCKET NO. 42144-502F01US], entitled “Implant With Outwardly Extending Fixation Elements,” filed on the same day herewith, and which is incorporated by reference in its entirety.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed. It should also be appreciated that although the devices, systems and methods described herein are described in reference to spinal procedures that they need not be limited to the spine. The devices, systems and method described herein can be used in non-spine procedures such as other orthopedic procedures, urological and gastroenterological procedures, or other non-spine procedures.

1. A surgical access and orthopedic distraction device, comprising:
a first cannula element comprising a first elongate portion and a semi-circular first head portion; and
a second cannula element comprising a second elongate portion and a semi-circular second head portion, wherein the first and second head portions reversibly mate to define a circular lip surrounding at least a portion of a proximal access portal of an elongate, tubular working channel formed by the first and second elongate portions; and
a first, rigid dilator element having an outer diameter larger than the inner diameter of the working channel, wherein the first, rigid dilator element is configured to be inserted through the working channel and separate the first cannula element a distance from the second cannula element and increase an inner diameter of the working channel to distract adjacent bones away from one another in at least a first direction.

2. The device of claim 1, further comprising a third elongate portion and a fourth elongate portion, wherein separation of the third elongate portion a distance from the fourth elongate portion enlarges the inner diameter of the working channel and distracts the adjacent bones away from one another in at least a second direction.

3. The device of claim 1, wherein the rigid dilator element is configured to increase an inner diameter of the working channel to distract a superior vertebral body and an inferior vertebral away from one another in the at least a first direction.

4. The device of claim 1, further comprising a second, rigid dilator element having an outer diameter larger than the inner diameter of the working channel and an inner diameter larger than the outer diameter of the first, rigid dilator element.

5. The device of claim 1, further comprising an expansion element mechanically coupled to one or both of the first head portion and the second head portion.

6. The device of claim 5, wherein the expansion element is configured to provide auxiliary separation of the first cannula element from the second cannula element.

7. The device of claim 1, further comprising a handle coupled to at least one of the first head portion or the second head portion and configured to manipulate the device.

8. The device of claim 1, wherein the first and second elongate portions have a U-shaped cross section and the working channel has a rectangular cross section.

9. The device of claim 1, wherein the lip surrounding at least a portion of the proximal access portal comprises a beveled edge.

10. The device of claim 1, wherein the first and second elongate portions are radiopaque.

11. A method of treating a spinal disorder in a patient, comprising:
accessing a disc space between an inferior vertebra and a superior vertebra with a trocar inserted through Kambin's triangle;
feeding an access device over the trocar to the disc space, wherein the access device comprises:
a first cannula element comprising a first elongate portion and a semicircular first head portion; and a second cannula element comprising a second elongate portion and a semicircular second head portion, wherein the first and second head portions reversibly mate to define a circular lip surrounding at least a portion of a proximal access portal of an elongate, tubular working channel formed by the first and second elongate portions;
sequentially advancing rigid dilator elements through the working channel of the access device;
separating the first elongate portion away from the second elongate portion;
pressing against the endplates of the inferior and superior vertebrae with a distal end region of the first and second elongate portions;
distracting the superior and inferior vertebrae away from one another with the distal end region of the first and second elongate portions; and
dilating tissues surrounding the first and second elongate portions of the access device.

12. The method of claim 11, wherein sequentially advancing rigid dilator elements comprises inserting a first, rigid dilator element through the working channel of the access device and inserting a second, rigid dilator element through the working channel over the first, rigid dilator element.

13. The method of claim 12, wherein the first, rigid dilator element has an outer diameter that is larger than an inner diameter of the working channel and wherein the second, rigid dilator element has an outer diameter larger than the inner diameter of the working channel and an inner diameter larger than the outer diameter of the first, rigid dilator element.

14. The method of claim 13, wherein inserting the first, rigid dilator element through the working channel separates the first and second elongate portions a distance away from one another.

15. The method of claim 14, wherein inserting the second, rigid dilator element through the working channel over the first, rigid dilator element enlarges the distance away the first and second elongate portions are separated from one another.

16. The method of claim 15, wherein enlarging the distance away the first and second elongate portions are separated from one another enlarges the inner diameter of the working channel.

17. The method of claim 11, further comprising using the distal end region of the first and second elongate portions to prepare the endplates for fusion.

18. The method of claim 11, further comprising delivering an intervertebral fixation device through the working channel of the access device to the disc space.

19. The method of claim 14, further comprising injecting bone growth stimulating material into an internal volume of the intervertebral fixation device.

20. A kit for use in accessing and distracting tissue, comprising:
an access and distraction device comprising:
a first cannula element comprising a first elongate portion and a semicircular first head portion; and
a second cannula element comprising a second elongate portion and a semicircular second head portion, wherein the first and second head portions reversibly mate to define a circular lip surrounding at least a portion of a proximal access portal of an elongate, tubular working channel formed by the first and second elongate portions; and
a first, rigid dilator element having an outer diameter larger than an inner diameter of the working channel, wherein the first, rigid dilator element is configured to be inserted through the working channel and separate the first cannula element a distance from the second cannula element and increase an inner diameter of the working channel to distract adjacent bones away from one another in at least a first direction.

21. The kit of claim 20, further comprising a second, rigid dilator element having an outer diameter larger than the inner diameter of the working channel and an inner diameter larger than the outer diameter of the first, rigid dilator element.

22. The kit of claim 20, further comprising an intervertebral fixation device deliverable through the working channel of the access device to a disc space.

23. The kit of claim 22, further comprising a delivery device coupled to the intervertebral fixation device.

24. The kit of claim 23, further comprising bone growth stimulating material.

25. The method of claim 18, wherein the intervertebral fixation device comprises:
a housing sized to be positioned between adjacent inferior and superior vertebrae, the housing comprising an internal volume defined by an inferior coupling element, a superior coupling element, and first and second sidewalls, wherein the internal volume comprises a longitudinal axis extending between the inferior coupling element and the superior coupling element and extending through the first and second sidewalls;
at least one flexible element coupled to an internal surface of at least one of the inferior or superior coupling elements, wherein the at least one flexible element projects inward toward the longitudinal axis of the internal volume of the housing when in a first configuration and projects outward away from the longitudinal axis of the internal volume toward the internal surface to which the at least one flexible element is coupled when in a second configuration; and
at least one fixation member coupled to the flexible element and sized to extend through a corresponding aperture in the inferior or superior coupling element.
